(19) United States
(12) Patent Application Publication
(10) Pub. No.: US 2008/0208312 A1
(43) Pub. Date: Aug. 28, 2008

(54) Stent Graft With Strips to Promote Localized Healing

(75) Inventors: Brian Kwitkin, Pembroke Pines, FL (US); Didier Billy, Maastricht (NL); Marc Hendriks, Brunssum (NL)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/464,585

(22) Filed: Aug. 15, 2006

Related U.S. Application Data
(60) Provisional application No. 60/713,776, filed on Sep. 2, 2005.

(51) Int. Cl. A61F 2/06

(52) U.S. Cl. 623/1.15; 623/1.13; 623/1.35

(57) ABSTRACT

An endoluminal stent graft includes segments of a healing promoter to promote the “healing in” of the distal and/or proximal neck(s) of the endoluminal stent graft in a vessel, thus reducing the risk of migration and the occurrence of endoleaks that can formed at the side of the neck(s) and the consequent feeding of the aneurysm sac. In some applications, the segments of the healing promoter are located within a proximal anchor region located near the proximal neck opening of the endoluminal stent graft and, optionally, within one or more distal anchor regions located near one or more distal neck openings of the endoluminal stent graft. In other applications, the segments of the healing promoter are located within the proximal anchor region, but not the distal anchor region.

### RELATED APPLICATIONS

This application claims the benefit of U.S. Provisional Patent Application 60/713,776 filed Sep. 2, 2005.

### BACKGROUND OF THE INVENTION

1. Field of the Invention

The present invention relates generally to stent grafts, and more particularly to improving healing associated with placement of an endoluminal stent graft in a vessel.

2. Description of the Related Art

Vascular aneurysms are the result of abnormal dilation of a blood vessel, usually resulting from disease and/or genetic predisposition, which can weaken the arterial wall and allow it to expand. While aneurysms can occur in any blood vessel, most occur in the aorta and peripheral arteries, with the majority of aortic aneurysms occurring in the abdominal aorta, usually beginning below the renal arteries and often extending distally into one or both of the iliac arteries.

Aortic aneurysms are often treated in open surgical procedures where the diseased vessel segment is bypassed and repaired with an artificial vascular graft. While considered an effective surgical technique, conventional vascular graft surgery however, is frequently not advisable for elderly patients or those patients weakened from cardiovascular and other diseases.

An alternative treatment to the open surgical procedure is placement of an endovascular prosthesis, such as an endoluminal stent graft, inside the vessel to isolate the aneurysm from blood flow and subsequent pressure. Generally, endoluminal stent grafts are delivered to a desired location within a vessel using a catheter-based delivery technique. To deliver the endoluminal stent graft within an acceptable size for a blood vessel, endoluminal stent grafts are typically compressed and housed in a removable sheathing. The endoluminal stent graft is then inserted into a vessel via the catheter-based delivery technique, positioned in the vessel, and the sheath removed allowing the endoluminal stent graft to expand and contact the vessel walls. Conventionally, the proximal end of the endoluminal stent graft is referenced with respect to the end closest to the heart (via the length of blood traveled from the heart). Some endoluminal stent grafts further include openings or side openings or are constructed with integral bifurcations to accommodate lateral branches off or branching of the main vessel.

Endoluminal stent grafts typically include a graft material attached to a stent structure. The graft material is generally formed into a tubular shape with a hollow lumen. The graft material is typically a material that channels blood through the graft lumen without excessive leakage of blood into the surrounding vessel, and thus the graft material is typically tightly woven.

The stent structure is attached to the graft material so that when the stent structure is expanded, the stent-graft forms a tubular shape. The stent structure is typically formed of stainless steel, nitinol or other materials capable of being expanded with the graft material to strengthen the walls of the vessel and/or to provide support for the graft material through the vessel, e.g., through the aneurysm section of the vessel. Some portions of the stent structure are attached at the ends of the graft material at the lumen openings to provide additional support or anchoring of the endoluminal stent graft in the vessel.

Unfortunately, prior art endoluminal stent grafts when implanted in some patients developed a number of technical problems with subsequent morbidity and/or mortality of the patient. In particular, the proximal neck of the prior art endoluminal stent grafts did not heal in well to the vessel wall. The lack of healing in and incorporation of the endoluminal stent graft at the aneurysm neck allowed the endoluminal stent graft to dislodge and migrate distally inside the aortic vessel permitting renewed feeding of blood and pressure to the aneurysm sac with the consequent risk of aneurysm rupture. Markedly affected were patients with severe neck angularity, e.g., those with an aortic neck shorter than 10 mm, due to insufficient contact surface with the vessel and insufficient anchoring force associated with the short neck.

### SUMMARY OF THE INVENTION

An endoluminal stent graft includes one or more segments of a healing promoter attached within a proximal anchor region of an endoluminal stent graft, and, optionally, within one or more distal anchor regions. When the endoluminal stent graft is positioned within a vessel, the segments of the healing promoter promote and guide the migration, proliferation and adhesion of vessel cells to the endoluminal stent graft to increase localized healing. Thus, healing time after implant of an endoluminal stent graft may be decreased and a more stable implant produced that is less susceptible to migration and/or endoleaks that could otherwise form at the sides of the proximal neck and the consequent feeding of the aneurysm sac.

### BRIEF DESCRIPTION OF THE DRAWINGS

FIG. 1 is a front schematic view that illustrates one example of an endoluminal stent graft including one or more segments of a healing promoter;

FIG. 2 is a top view of the endoluminal stent graft of FIG. 1;

FIG. 3 is a side schematic view that illustrates one example of a portion of a segment of a healing promoter formed of a healing promoter including one or more loop-like structures and one or more tail-like structures;

FIG. 4 is a front schematic view that illustrates another example of an endoluminal stent graft including one or more segments of a healing promoter;

FIG. 5 is a top view of the endoluminal stent graft of FIG. 4;

FIG. 6 is a front schematic view that illustrates yet another example of an endoluminal stent graft including one or more segments of a healing promoter;

FIG. 7 illustrates a top view of the endoluminal stent graft of FIG. 6; and

FIG. 8 is a cross sectional schematic view that illustrates one example of an endoluminal stent graft including one or more segments of a healing promoter positioned within a vessel.

Common reference numerals are used throughout the drawings and detailed description to indicate like elements.

### DETAILED DESCRIPTION

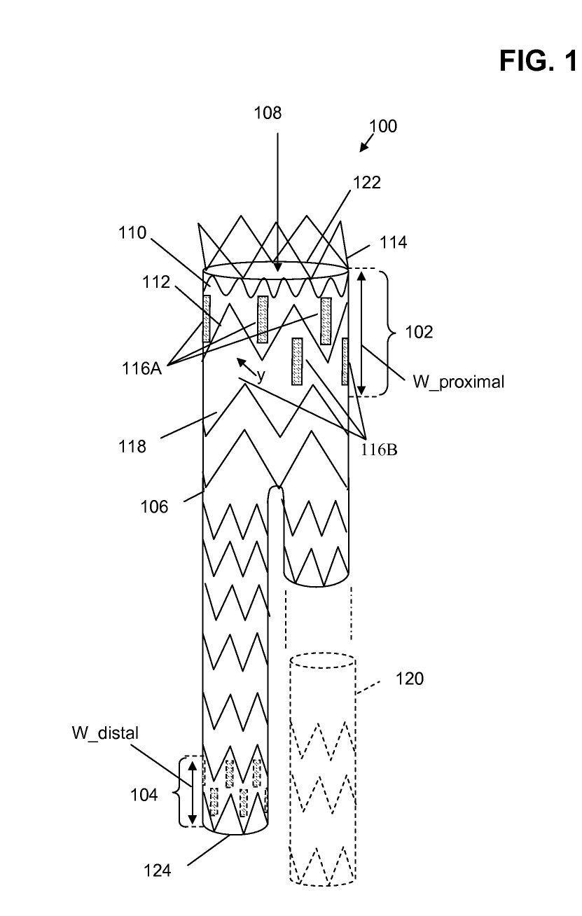

FIG. 1 illustrates one example of an endoluminal stent graft 100 including one or more segments 116A, 116B of a healing promoter. In FIG. 1, endoluminal stent graft 100, herein termed simply stent graft 100, includes: a graft material 106, i.e., a first material; one or more segments 116A, 116B of a healing promoter, i.e., one or more segments of a second material, attached to graft material 106; and a stent structure of springs attached to graft material 106, including a first (base) spring 110, a second (support) spring 112, an anchor spring 114, and other springs, such as support spring 118. As illustrated in FIG. 1, stent graft 100 is shaped to form a lumen 108 that bifurcates distally to accommodate branching of the aorta into smaller downstream vessels, e.g., the common iliac arteries. In some stent graft configurations, an extension 120 is included as part of main stent graft body 100.

In one example, graft material 106 is a material formed to limit the leakage of blood through graft material 106. Examples of graft material 106 include substantially non-porous fabrics, such as low profile system (LPS) material (a woven monofilament polyester), reduced porosity material (RPM) material a woven polyester material, or densely knitted fabrics (such as HDM—a High Density Material—a more tightly woven polyesther material. Any of the commonly used graft materials are suitable for use herein. The stent structure can be attached to the exterior side of graft material 106 or to the internal side, i.e., the luminal side, of graft material 106, or both.

Segments 116A, 116B of the healing promoter are attached to portions of an exterior circumferential surface of graft material 106 within a proximal anchor region 102 located at a proximal neck of stent graft 100. In this example, proximal anchor region 102 extends from a proximal circumferential edge 122 longitudinally towards the distal end of stent graft 100 a specified distance W_proximal. In one example, specified distance W_proximal defines a length of what is commonly referred to as the proximal neck of stent graft 100. Thus, a group of stent grafts is provided having a range of specified distances W_proximal so that the range of specified distances corresponds to the range of aneurysm necks commonly encountered in patients. A physician chooses a particular stent graft in the group based on the characteristics of the aneurysm neck in a particular patient. The actual dimension associated with W_proximal will in the range of 5 to 40 mm.

In this example, segments 116A, 116B of the healing promoter are formed as rectangular strips. More specifically, each segment is formed in a rectangular shape having two substantially parallel shorter sides and two substantially parallel longer sides, the two substantially parallel longer sides are oriented substantially perpendicular to proximal circumferential edge 122 of endoluminal stent graft 100. Here, substantially means within manufacturing tolerances for a particular healing promoter. The range of sizes is such that alternating regions of healing promoter and bare graft material provide space for the combination structure to be readily compressed to a small diameter at which the compressed combination is held by a surrounding delivery sheath.

The non-continuous nature and spacing of the strips allows for the folding of the segments 116A, 116B to alternately face one another as they are compressed to be held by the sheath of a delivery catheter.

However, in other examples (not shown), segments 116A, 116B of the healing promoter are formed in other shapes including, but not limited to, triangular, circular, square, oval, and trapezoidal. Further, although segments 116A, 116B of the healing promoter are illustrated two-dimensionally, segments of the healing promoter have a thickness which can be varied dependent upon the properties of the healing promoter from which segments 116A, 116B of the healing promoter are formed and/or to promote desired healing properties.

Irrespective of their shape, segments 116A, 116B of the healing promoter are positioned to facilitate the migration, proliferation and adhesion of vessel cells to stent graft 100 when stent graft 100 is positioned in the vessel. In FIG. 1, each of the rectangular segments 116A, 116B of the healing promoter is positioned such that a side y is oriented substantially parallel to the longitudinal axis of stent graft 100 when stent graft 100 is deployed. Herein, the use of the term substantially indicates a close approximation to a desired parameter but does not require an exact adherence to the desired parameter. For example, a substantially parallel need not be exactly parallel, but rather a close approximation to parallel given the manufacturing tolerances and limitations imposed by the environment in which stent graft 100 is used.

Further, segments 116A, 116B of the healing promoter 116, in this example, are attached to graft material 106 in a substantially, evenly spaced arrangement around the circumferential outer surface of stent graft 100 within proximal anchor region 102. In FIG. 1, segments 116A of the healing promoter are attached in a substantially, evenly spaced arrangement between first (base) spring 110 and second (support) spring 112. Similarly, segments 116B of the healing promoter are attached in a substantially, evenly spaced arrangement between second (support) spring 112 and support spring 118. Segments 116B of the healing promoter positioned between second (support) spring 112 and support spring 118 are offset relative to segments 116A of the healing promoter positioned between first (base) spring 110 and second (support) spring 112.

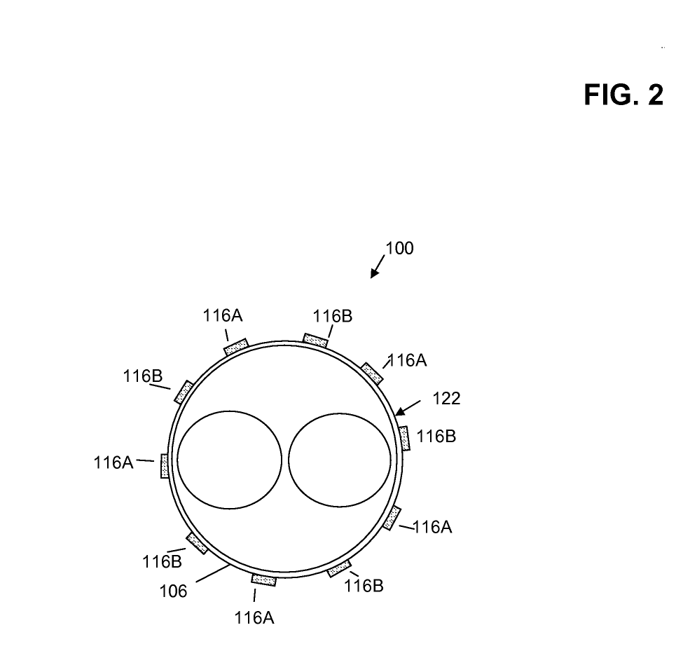

FIG. 2 illustrates a cross-sectional view of anchor region 102 of endoluminal stent graft 100. In FIG. 2, segments 116A, 116B of the healing promoter are shown substantially, evenly spaced around the outer circumferential surface of stent graft 100. The two sets of offset segments 116A, 116B of the healing promoter provide a substantially evenly spaced circumferential distribution of locations around the outer circumferential surface of stent graft 100 where vessel cell adhesion can occur.

Each of segments 116A, 116B of the healing promoter is formed of a healing promoter, as described more completely below, that supports cellular in growth and consequent fixation of an endoluminal stent graft, such as stent graft 100, within a vessel. The attachment of segments 116A, 116B of the healing promoter in proximal anchor region 102 promotes healing in of the proximal neck of stent graft 100 in a vessel, thus reducing the risk of dislodgement and distal migration, and the occurrence of endoleaks that could otherwise form at the side of the proximal neck and the consequent feeding of the aneurysm sac.

Referring again to FIG. 1, optionally, segments of the healing promoter are attached to portions of the outer circumferential surface of graft material 106 within a distal anchor region 104 located at a distal neck (or necks) of stent graft 100. Distal anchor region 104 extends from a distal circumferential edge 124 longitudinally towards the proximal end of stent graft 100 a specified distance W_distal. In one example, segments of the healing promoter are positioned between springs in distal anchor region 104 similar to the positioning of segments 116A, 116B of the healing promoter within proximal anchor region 102.

In one example, healing promoter segments 116A, 116B are made of a porous fabric, such as a Dacron fabric, or non-woven material whose porosities are greater than the porosity of the underlying graft material itself. So that tissue and cells which are likely to form an anchoring framework to the surrounding vessel, are easily received and attach themselves to the segments 116A, 116B.

In one example, healing promoter segments 116A, 116B include a coating on a material that further promotes healing-in, such as a collagen coating. In another example, healing promoter segments 116A, 116B include at least one growth factor promoting agent, such as a ReGeneraTing Agent (RGTA). RGTA is chemically substituted dextran. RGTA is encapsulated in a material forming healing promoter segments 116A, 116B, or alternatively is applied directly to the material forming healing promoter segments 116A, 116B, for example, as a coating. Whose manipulation and use as a coating is understood by a person skilled in the art.

Alternatively, rather than using a material, healing promoter segments 116A, 116B are a coating directly on graft material 106. In one example, healing promoter segments 116A, 116B are a drug-impregnated coating that promotes the formation of thrombosis and tissue incorporation between stent graft 100 and a vessel. In another example, healing promoter segments 116A, 116B are coated on graft material 106, portions of the stent structure, such as any of first (base) spring 110, second (support) spring 112, support spring 118, and anchor spring 114, among others, or both.

In one example, the drug-impregnated coating is a drug impregnated polymer coating, such as polyvinyl alcohol or polyethylene glycol.

In another example, the drug-impregnated coating is an adhesive, which activates on contact with blood. For example, the adhesive is of the type that increases in size, i.e., swells, when in contact with blood.

In yet another example, the drug impregnated coating includes at least one drug clotting factor and at least one drug tissue attachment factor. The at least one drug clotting factor is selected from a group consisting of clotting factors I, II, III, IV, V, VI, VII, and VIII, thrombin, and fibrinogen. The at least one drug tissue attachment factor is selected from a group consisting of vitronectin, fibronectin, laminin, and a sclerosing agent. In some applications, the at least one drug tissue attachment factor is slow releasing.

In one example, the drug-impregnated coating includes at least one growth factor promoting agent, such as ReGeneraTing Agent (RGTA).

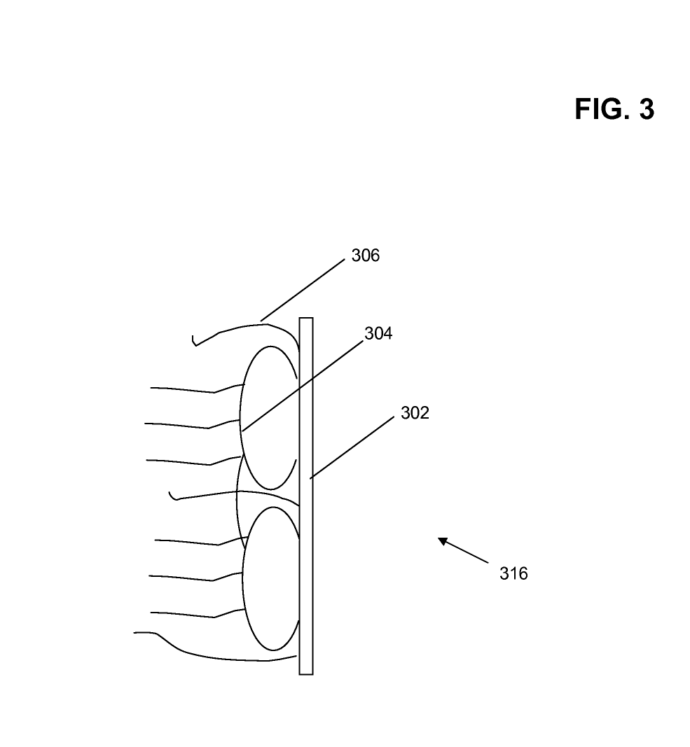

In yet another example, the porous fabric of the healing promoter segments 116A, 116B includes a loop-like structure, a tail-like structure, or both to promote to promote tissue incorporation, the formation of thrombosis, and fixation of an endoluminal stent graft, such as endoluminal stent graft 100, in a vessel as further described herein with reference to FIG. 3.

FIG. 3 illustrates one example of a portion of a segment 316 of a healing promoter formed of a porous fabric including one or more loop-like structures 304 and one or more tail-like structures 306. Each segment, or alternatively selected segments, of the healing promoter includes a support material 302 having one or more loop-like structures 304, herein termed loops 304, and one or more tail-like structures 306, herein termed tails 306, attached.

In one example, loops 304, tails 306, or both include at least one drug, for example, at least one drug-clotting factor. The at least one drug clotting factor is selected from the group consisting of clotting factors I, II, III, IV, V, VI, VII, and VIII, thrombin, and fibrinogen.

In another example, loops 304, tails 306, or both include at least one drug tissue attachment factor selected from a group consisting of vitronectin, fibronectin, laminin, and sclerosing agent. Examples of a sclerosing agent include morrhuate sodium, ethanolamine oleate, and tetradecyl sulfate. In one application, the drug tissue attachment factor is slow releasing.

In yet another example, loops 304, tails 306, or both are made of a biocompatible copolymer. For example, loops 304, tails 306, or both are made of polyester, such as Dacron or polytetrafluoroethylene (PTFE).

In one application, loops 304, tails 306, or both are attached to support material 302 by sewing or weaving. In another application, loops 304, tails 306, or both are attachable directly to graft material 106, the stent structure, such as any of first (base) spring 110, second (support) spring 112, and anchor spring 114, among others, or both to promote tissue incorporation and the fixation of a stent graft, such as stent graft 100, in a vessel. In one example, loops 304, tails 306, or both swell when in contact with blood.

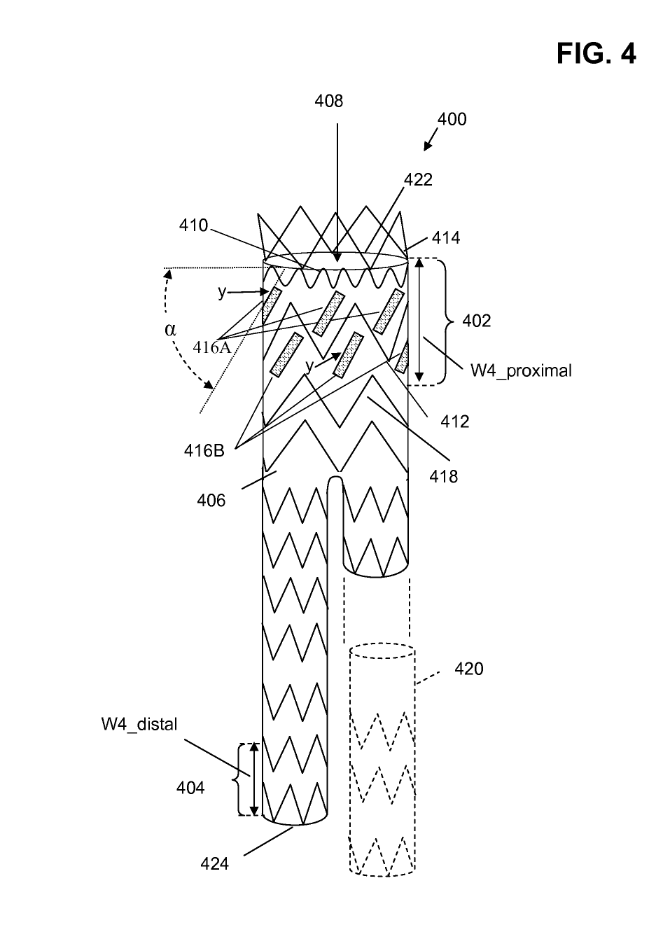

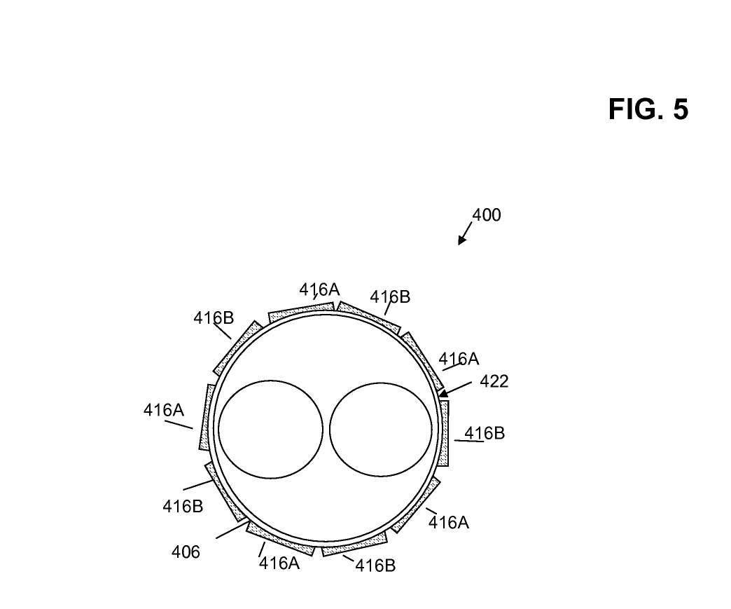

Stent graft 100 illustrates segments 116A, 116B of the healing promoter formed in a rectangular shape and attached to graft material 106 in an orientation such that side y is substantially parallel to the longitudinal axis of stent graft 100. In other examples, segments of the healing promoter can be attached to graft material 106 in different orientations, such as at an angle to proximal circumferential edge 122, as further described herein with reference to FIGS. 4 and 5.

FIG. 4 illustrates one example of an endoluminal stent graft 400 including one or more segments 416A, 416B of a healing promoter. Endoluminal stent graft 400, herein termed simply stent graft 400, includes: a graft material 406, i.e., a first material; one or more segments 416A, 416B of the healing promoter, e.g., one or more segments of a second material, attached to graft material 406; and a stent structure of springs attached to graft material 406, such as a first (base) spring 410, a second (support) spring 412, an anchor spring 414, and other springs, such as support spring 418.

As illustrated in FIG. 4, stent graft 400 is shaped to form a lumen 408 that bifurcates distally to accommodate lateral vessels, e.g., the common iliac arteries. In some applications, an extension 420 is included as part of stent graft 400. Graft material 406 is a material that limits the leakage of blood through graft material 406, such as those materials earlier described with reference to stent graft 100 and graft material 106 (FIG. 1).

Segments 416A, 416B of the healing promoter are attached to portions of the outer circumferential surface of graft material 406 within a proximal anchor region 402 located at the proximal neck of stent graft 400. Proximal anchor region 402 extends from a proximal circumferential edge 422 longitudinally towards the distal end of stent graft 400 a specified distance W4_proximal. The stent structure can be attached to the exterior side of graft material 406, to the internal side of graft material 106, or both.

Segments 416A, 416B of the healing promoter are formed as rectangular strips. Again, in other examples, segments 416A, 416B of the healing promoter are formed in other shapes including, but not limited to, triangular, circular, square, oval, and trapezoidal shapes. Further, although segments 416A, 416B of the healing promoter are illustrated two-dimensionally, segments 416A, 416B of the healing promoter have a thickness, which can be varied dependent upon the properties of the healing promoter from which segments 416A, 416B are formed and/or to promote desired healing properties.

Each of segments 416A, 416B of the healing promoter are positioned to guide the migration, proliferation and adhesion of vessel cells to stent graft 400 when stent graft 400 is positioned in a vessel. In FIG. 4, each of segments 416A, 416B of the healing promoter is positioned such that a side y of each of segments 416A, 416B of the healing promoter is oriented at an angle α to proximal circumferential edge 422. Angle α is in a range from about 0 degrees, e.g., about parallel to edge 422, to about 180 degrees. In the example of FIG. 4, angle α, is about 45 degrees, while in FIGS. 1 and 3, angle α is about 90 degrees

Further, segments 416A, 416B of the healing promoter are attached to graft material 406 in a substantially, evenly spaced arrangement around the outer circumferential surface of stent graft 400 within proximal anchor region 402. In FIG. 4, segments 416A of the healing promoter are attached in a substantially, evenly spaced arrangement between first (base) spring 410 and second (support) spring 412. Similarly, segments 416B of the healing promoter are attached in a substantially, evenly spaced arrangement between second (support) spring 412 and support spring 418. Segments 416B of the healing promoter positioned between second (support) spring 412 and support spring 418 are offset relative to segments 416A of the healing promoter positioned between first (base) spring 410 and second (support) spring 412.

FIG. 5 illustrates a cross-sectional distal view of endoluminal stent graft 400. In FIG. 5, segments 416A, 416B of the healing promoter are shown substantially, evenly spaced around the outer circumferential surface of stent graft 400. The two sets of offset segments 416A, 416B provide a substantially evenly spaced circumferential distribution of locations where vessel cell adhesion can occur. In angling the orientation of each of segments 416A, 416B of the healing promoter (relative to proximal circumferential edge 422), a more continuous distribution of locations where vessel cell adhesion can occur around stent graft 400 is provided.

Referring again to FIG. 4, optionally, segments of the healing promoter are attached to portions of the outer circumferential surface of graft material 406 within a distal anchor region 404 located at a distal neck (or necks) of stent graft 400. Distal anchor region 404 extends from a distal circumferential edge 424 longitudinally towards the proximal end of stent graft 400 a specified distance W4_distal. In one example, segments of the healing promoter are positioned between springs in distal anchor region 404 similar to the positioning of segments 416A, 416B of the healing promoter within proximal anchor region 402.

Each of segments 416A, 416B of the healing promoter is formed of a healing promoter, such as any of the healing promoters earlier described with reference to segments 116A, 116B of the healing promoter and stent graft 100 (FIG. 1). Stent grafts 100 and 400 illustrate examples of endoluminal stent grafts in which segments of the healing promoter are attached to a graft material within a proximal anchor region (and optionally within a distal anchor region) and are located on the exterior circumferential surface of the graft material between portions of the stent structure, such as between springs.

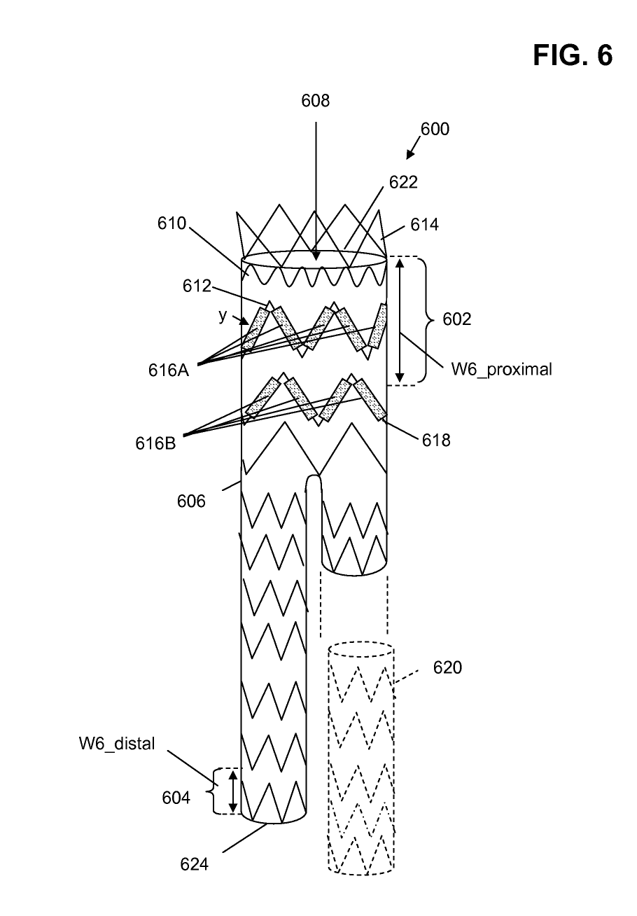

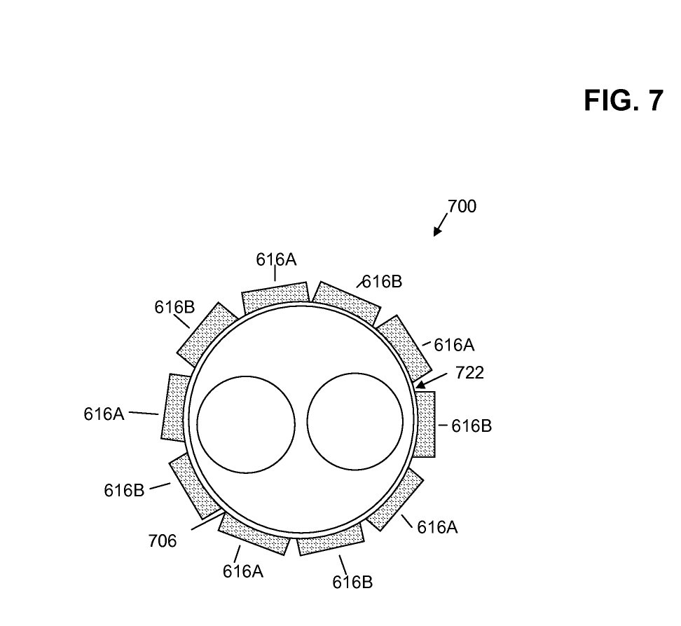

As earlier described, typically, the stent structure of an endoluminal stent graft is expanded within a vessel until contact is made with the vessel wall. As the stent structure typically provides the initial anchoring force against the vessel wall, segments of the healing promoter also are attached to a graft material and cover portions of the stent structure to allow better contact with a vessel wall as further described herein with reference to FIGS. 6 and 7.

FIG. 6 illustrates one example of an endoluminal stent graft 600 including one or more segments 616A, 616B of a healing promoter. In FIG. 6, endoluminal stent graft 600, herein termed simply stent graft 600, includes: a graft material 606, i.e., a first material; one or more segments 616A, 616B of the healing promoter, e.g., one or more segments of a second material, attached to graft material 606; and a stent structure of springs attached to graft material 606, such as a first (base) spring 610, a second (support) spring 612, an anchor spring 614, and other springs, such as support spring 616.

As illustrated in FIG. 6, stent graft 600 is shaped to form a lumen 608 that bifurcates distally to accommodate lateral vessels, e.g., the common iliac arteries. In some applications, an extension 620 is included as part of stent graft 600. Graft material 606 is a material that limits the leakage of blood through graft material 606, such as those materials earlier described with reference to stent graft 100 and graft material 106 (FIG. 1).

Segments 616A, 616B of the healing promoter are attached to portions of the outer circumferential surface of graft material 606 within a proximal anchor region 602 located at the proximal neck of stent graft 600 and cover portions of the stent structure, e.g., second (support) spring 612 and support spring 618. Proximal anchor region 602 extends from a proximal circumferential edge 622 toward the distal end of stent graft 600 a specified distance W6_proximal. The stent structure can be attached to the exterior side of graft material 606, to the internal side of graft material 606, or both.

Segments 616A, 616B of the healing promoter are formed as rectangular strips. However, segments of the healing promoter can be formed in other shapes including, but not limited to, triangular, circular, square, oval, and trapezoidal shapes. Further, although segments 616A, 616B of the healing promoter are illustrated two-dimensionally, segments 616A, 616B of the healing promoter have a thickness which can be varied dependent upon the properties of the healing promoter from which segments 616A, 616B are formed and/or to promote desired healing properties.

Each of segments 616A, 616B of the healing promoter is positioned to guide the migration, proliferation and adhesion of vessel cells to stent graft 600 when stent graft 600 is positioned in a vessel. In FIG. 6, each of segments 616A, 616B of the healing promoter is positioned over a portion of the stent structure, e.g., with a side y substantially parallel to and covering a portion of second (support) spring 612 and support spring 618, as further described herein with reference to FIG. 7.

FIG. 7 illustrates a cross-sectional distal view of endoluminal stent graft 600. In FIG. 7, segments 616A, 616B of the healing promoter are shown attached to portions of graft material 606 and covering portions of second (support) spring 612 and support spring 618 (not shown). By positioning segments 616A, 616B of the healing promoter over second (support) spring 612 and support spring 618 within distal anchor region 602, segments 616A, 616B of the healing promoter are in direct forceful direct contact with the vessel wall when the stent structure of endoluminal stent graft 600 is expanded within the vessel until contact is made with the vessel wall.

Referring again to FIG. 6, optionally, in some applications, segments of the healing promoter are attached to portions of the exterior circumferential surface, of graft material 606 and covering portions of the stent structure within a distal anchor region 604 located at a distal neck (or necks) of stent graft 600. Distal anchor region 604 extends from a distal circumferential edge 624 longitudinally towards the proximal end of stent graft 600 a specified distance W6_distal. The segments of the healing promoter are positioned covering portions of the stent structure in distal anchor region 604 similar to the positioning of segments 616A, 616B of the healing promoter within proximal anchor region 602.

In one example, each of segments 616A, 616B of the healing promoter is formed of a healing promoter that supports cellular in growth and consequent fixation of an endoluminal stent graft, such as stent graft 600, within a vessel, such as any of the healing promoters earlier described with reference to segments 116A, 116B of the healing promoter and stent graft 100 (FIG. 1). The attachment of segments 616A, 616B of the healing promoter in proximal anchor region 602 promotes healing in of the proximal neck of stent graft 600 in a vessel reducing the risk of dislodgement and distal migration, thus reducing the occurrence of endoleaks that could otherwise form at the side of the proximal neck and the consequent feeding of the aneurysm sac.

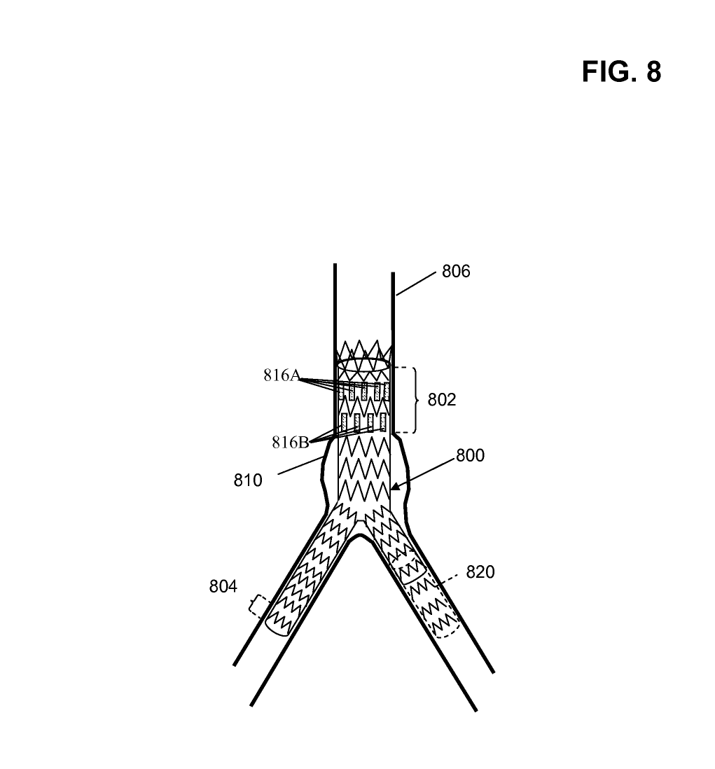

FIG. 8 illustrates one example of an endoluminal stent graft 800 including a plurality of segments 816A, 816B of the healing promoter located within a proximal anchor region 802. In FIG. 8, endoluminal stent graft 800 is illustrated positioned within vessel 806 spanning aneurysmal sac 810, for example, using a catheter-based delivery technique, such that segments 816A, 816B of the healing promoter contact the walls of vessel 806 in proximal anchor region 804.

Segments 816A, 816B of the healing promoter are positioned to guide the migration, proliferation and adhesion of vessel cells to stent graft 800. Each of segments 816A, 816B of the healing promoter is oriented similar to segments 116A, 116B of the healing promoter of stent graft 100 (FIG. 1). Each of segments 816A, 816B of the healing promoter is formed of a healing promoter that supports cellular in growth and consequent fixation of endoluminal stent graft 800 and extension 820 within vessel 806, such as any of the healing promoters earlier described with reference to segments 116A, 116B of the healing promoter and stent graft 100 (FIG. 1).

This disclosure provides exemplary examples of the present invention. The scope of the present invention is not limited by these exemplary examples. In particular, while segments 116A, 116B 416A, 416B 616A, 616B, 816A, 816B of the healing promoter are primarily illustrated as located in a distal anchor region, segments of the healing promoter can also be located in one or more proximal anchor regions. Also, although a set of segments of the healing promoter is illustrated using a single reference numeral, a set of segments of the healing promoter need not be of a same healing promoter, and can be from different healing promoters. For example, segments of a healing promoter may be a fabric in a distal or proximal anchor region and a coating in the other region, or a mix within each region. Further, segments of a healing promoter may be of different fabrics or different coatings within a distal and/or a proximal anchor region. Additionally, it may be desirable to include segments of the healing promoter on a stent graft extension. Also, although segments of the healing promoter are primarily illustrated in an evenly spaced arrangement around a respective stent graft, different spacing arrangements can be used to provide a desired healing in effect.

Thus, numerous variations, whether explicitly provided for by the specification or implied by the specification or not, such as variations in structure, dimension, type of material and manufacturing process may be implemented by one of skill in the art in view of this disclosure. The above detailed description is illustrative of an endoluminal stent graft having a bifurcated structure, however, the invention is not limited thereto and is applicable to a wide variety of endoluminal stent graft designs, including other bifurcated and non-bifurcated designs, as well as other stent structures, including other spring structures, strut structures, interwoven structures, and interlocking structures, among others.

What is claimed is:

1. An endoluminal stent graft having a proximal neck and at least one distal neck comprising:
a graft material;
a stent structure attached to said graft material; and
at least one segment of a healing promoter located within a proximal anchor region of said proximal neck wherein said healing promoter is a material that supports cellular in growth and consequent fixation of said endoluminal stent graft in a vessel.

2. The endoluminal stent graft of claim 1, wherein said at least one segment of said healing promoter covers a portion of said stent structure.

3. The endoluminal stent graft of claim 1, wherein said at least one segment of a healing promoter is attached to an exterior side of said graft material.

4. The endoluminal stent graft of claim 2 further comprising:
another at least one segment of said healing promoter located within a distal anchor region of said distal neck.

5. The endoluminal stent graft of claim 1, wherein said at least one segment of said healing promoter is formed in a rectangular shape having two substantially parallel shorter sides and two substantially parallel longer sides, said two substantially parallel longer sides being oriented substantially perpendicular to a proximal circumferential edge of said endoluminal stent graft.

6. The endoluminal stent graft of claim 1, wherein said at least one segment of said healing promoter is formed in a rectangular shape having two substantially parallel shorter sides and two substantially parallel longer sides, said two substantially parallel longer sides being oriented at an angle to a proximal circumferential edge of said endoluminal stent graft.

7. The endoluminal stent graft of claim 1 wherein said at least one segment of a healing promoter covers a portion of said stent structure.

8. The endoluminal stent graft of claim 1 wherein said healing promoter comprises:
a porous fabric.

9. The endoluminal stent graft of claim 8, wherein the porous fabric is a Dacron fabric.

10. The endoluminal stent graft of claim 1 wherein said healing promoter comprises:
at least a coating.

11. The endoluminal stent graft of claim 10 wherein said coating comprises a collagen coating.

12. The endoluminal stent graft of claim 10 wherein said coating comprises a drug impregnated coating that promotes formation of thrombosis and tissue incorporation between the endoluminal stent graft and a vessel.

13. The endoluminal stent graft of claim 12 wherein said drug impregnated coating comprises at least a drug impregnated polymer.

14. The endoluminal stent graft of claim 13, wherein said drug impregnated polymer is selected from a group consisting of polyvinyl alcohol and polyethylene glycol.

15. The endoluminal stent graft of claim 12, wherein said drug impregnated coating is hydrophilic.

16. The endoluminal stent graft of claim 12, wherein said drug impregnated coating comprises at least an adhesive, which activates on contact with blood.

17. The endoluminal stent graft of claim 12, wherein said drug impregnated coating increases in size when in contact with blood.

18. The endoluminal stent graft of claim 12, wherein said drug impregnated coating includes at least one drug clotting factor and at least one drug tissue attachment factor.

19. The endoluminal stent graft of claim 18, wherein said at least one drug clotting factor is selected from a group consisting of clotting factors I, II, III, IV, V, VI, VII, and VII, thrombin, and fibrinogen.

20. The endoluminal stent graft of claim 18, wherein said at least one drug tissue attachment factor is selected from a group consisting of vitronectin, fibronectin, laminin, and sclerosing agent.

21. The endoluminal stent graft of claim 18, wherein said at least one drug tissue attachment factor is a slow releasing drug tissue attachment factor.

22. The endoluminal stent graft of claim 1, wherein said healing promoter comprises:
at least a healing promoting agent.

23. The endoluminal stent graft of claim 22, wherein said healing promoting agent is selected from a group consisting of a growth factor, a hormone, an antibiotic, an immuno-suppressant, and a gene-containing product.

24. The endoluminal stent graft of claim 1 wherein said healing promoter comprises:
at least one loop-like structure.

25. The endoluminal stent graft of claim 24 wherein said healing promoter further comprises:
at least one tail-like structure.

26. The endoluminal stent graft of claim 1 wherein said healing promoter further comprises:
at least one tail-like structure.

27. The endoluminal stent graft of claim 25, wherein said at least one loop-like structure and said at least one tail-like structure swell when in contact with blood.

28. The endoluminal stent graft of claim 25, wherein said at least one loop-like structure and said at least one tail-like structure include at least one drug.

28. The endoluminal stent graft of claim 28, wherein said at least one drug is a drug clotting factor.

29. The endoluminal stent graft of claim 28, wherein said at least one drug is a drug tissue attachment factor.

30. The endoluminal stent graft of claim 29, wherein said drug tissue attachment factor is slow releasing.

31. The endoluminal stent graft of claim 25, wherein said at least one loop-like structure and said at least one tail-like structure are formed of at least one material selected from a group consisting of a biocompatible polymer, a biocompatible copolymer, a polyester, Dacron, and PTFE.
